(19) United States
(12) Patent Application Publication
(10) Pub. No.: US 2020/0345275 A1
(43) Pub. Date: Nov. 5, 2020

(54) A METHOD FOR MEASURING A SEDATION STATE OF A PATIENT

(71) Applicants: UNIVERSITE D'AIX-MARSEILLE (AMU), Marseille (FR); ASSISTANCE PUBLIQUE - HÔPITAUX DE MARSEILLE (AP-HM), Marseille (FR); INSTITUT FRANCAIS DES SCIENCES ET TECHNOLOGIES DES TRANSPORTS DE L'AMÉNAGEMENT ET DES RÉSEAUX, Marne-la-Vallée (FR); CENTRE NATIONAL DE LA RECHERCHE SCIENTIFIQUE (CNRS), Paris (FR)

(72) Inventors: Harold Noble Ibouanga-Kipoutou, Luynes (FR); Salah Boussen, Marseille (FR); Kouider Nacer M'Sirdi, Marseille (FR); Pierre-Jean Arnoux, Simiane Collongue (FR); Nicolas Bruder, Marseille (FR); Michel Behr, Marseille (FR)

(73) Assignees: UNIVERSITE D'AIX-MARSEILLE (AMU), Marseille (FR); ASSISTANCE PUBLIQUE - HOPITAUX DE MARSEILLE (AP-HM), Marseille (FR); INSTITUT FRANCAIS DES SCIENCES ET TECHNOLOGIES DES TRANSPORTS DE L'AMENAGEMENT ET DES RESEAUX, Marne-la-Vallée (FR); CENTRE NATIONAL DE LA RECHERCHE SCIENTIFIQUE (CNRS), Paris (FR)

(21) Appl. No.: 16/964,729

(22) PCT Filed: Jan. 25, 2019
(86) PCT No.: PCT/EP2019/051903

§ 371 Date: Jul. 24, 2020

(30) Foreign Application Priority Data
Jan. 26, 2018 (EP) 18153570.9

(51) Int. Cl. A61B 5/11; A61M 16/01; A61B 5/0205; G16H 20/40; G16H 50/30; G16H 40/63

(52) U.S. Cl. CPC A61B 5/1106; A61M 16/01; A61B 5/0205; G16H 20/40; A61B 5/0245; G16H 40/63; A61M 2230/06; A61M 2230/30; A61M 2230/42; G16H 50/30

(57) ABSTRACT

The invention relates to a method for automatic and continuous measurement of a sedation state of a patient in an intensive care unit. The method according to the invention comprises the following steps: providing signals representative of the condition of the patient, said signals comprising cardio-circulatory signals, signals representative of the respiratory activity and signals representative of the motor activity of the patient; and determining a global index representative of the sedation state of the patient.

### FIELD OF THE INVENTION

The present invention relates to a method for automatically and continuously measuring a sedation state of a patient in an intensive care unit.

### PRIOR ART

Upon admission in an intensive care unit, a patient is quickly monitored by a set of sensors, which allow to appreciate his or her clinical state, the evolution of this state, and the detection of vital or non-life-threatening emergencies. Thriving on the set of sensors, numerous physiological measures are collected, among which the electrocardiogram, the blood pressure, the respiratory volumes mobilized, as well as the adaptation of the patient to the ventilator (cough and overpressure, spontaneous volume, respiratory rate) if intubated.

Pharmacological sedation is essential for a large number of patients admitted in an intensive care unit. This sedation allows to limit the agitation of the patients and, consequently, the risks of removal of invasive devices such as intubation probes and catheters. It also allows adaptation to artificial ventilation, treating pain and improving the comfort of patients in a situation of intense stress.

Sedation consists in administering to the patient hypnotic and morphinic agents, which are intended to avoid agitation and its associated risks, to adapt the patient to the respirator in order to treat hypoxia, and to allow treatment gestures in good conditions of safety and comfort for the patient. These hypnotic and morphinic drugs have an effect on the above-mentioned physiological parameters. On the other hand, it is clear that an insufficiently sedated and agitated patient also sees its physiological parameters disturbed, for example, by an acceleration of the heart and respiratory rate and by an increase of the blood pressure and exhaled CO₂.

Numerous studies have shown the deleterious effects caused by a too deep or prolonged sedation. These effects include the extension of the artificial ventilation and associated complications, the increase of length of stay in intensive care units, an increase in the incidence of the cognitive dysfunctions during sedation and, in the long term, an excess of mortality.

The use of clinical sedation scores and sedation protocols based on these scores have been shown to reduce the duration of artificial ventilation, occurrence of intensive care unit (ICU) delirium, and to decrease the psychological after-effects of hospitalization. The monitoring of sedation using a score such as, for example, the Richmond Agitation Sedation Scale (RASS) has thus been generalized. Such clinical scores are routinely measured every 1 to 4 hours with an inter-individual variability in the assessment, which depends on the caregiver's experience and his or her level of training.

A score that would not depend on the observer and would be available on an ongoing basis would certainly allow a more precise steering of the sedation and a better evaluation of the practices. A reduction in the side effects of sedation would be expected as compared to current practices.

There is currently no device for reliable and objective measurement of the depth of sedation for patients in intensive care, independently of the observer. Indeed, the patent document WO201476356 describes an approach using electroencephalographic (EEG) sensors to evaluate the depth of sedation. However, this approach is limited because sedation does not have an EEG definition and the neurophysiological difference between wakefulness and sedation remains indistinct.

### SUMMARY OF THE INVENTION

Accordingly, a need exists for providing a method enabling an automated objective measurement of the depth of sedation of a patient in an intensive care unit, making it possible to solve the problems encountered in the prior art. This method aims to develop and validate an index of the depth of sedation of a patient, based on an automated analysis of collected physiological parameters, thereby allowing to track sedative states of a patient in real time.

In accordance with a first aspect, the invention concerns a method for automatic and continuous measurement of a sedation state of a patient in intensive care comprising the following steps:

providing signals representative of the condition of the patient, said signals comprising and, more particularly, consisting of, cardio-circulatory signals, signals representative of the respiratory activity and signals representative of the motor activity of the patient; and

determining a global index representative of the sedation state of the patient.

Thus, the method according to the invention makes it possible in particular to develop and validate the index of the depth of sedation of a patient, based on the automated analysis of the cardiovascular, respiratory and motor activity parameters. More particularly, the real-time evolution of these three parameters makes it possible to obtain a global sedation index (GSI). This index may then be quantified, in order to make the patient better suited to the environment of the intensive care unit.

Advantageously, the method according to the present invention is characterized in that: —it comprises the following steps: providing a multiparameter surveillance monitor centralizing measurements of various physiological variables of the patient including electrocardiogram measurements; providing signals representative of the condition of the patient, said signals comprising cardio-circulatory signals, signals representative of the respiratory activity and signals representative of the motor activity of the patient; and determining a global index representative of the sedation state of the patient, wherein the cardio-circulatory signals are provided by the multiparameter surveillance monitor, the signals representative of the respiratory activity of the patient are provided by a ventilator, and the signals representative of the motor activity are provided by means for measuring motor activity of the patient—it further comprises the steps of: calculating, from the signals representative of the condition of the patient, at least one index calculated from the cardio-circulatory signals, at least one index calculated from the signals representative of the respiratory activity, and at least one index representative of the motor activity of the patient; and determining, from these indexes, the global index representative of the sedation state of the patient; —the at least one index calculated from the cardio-circulatory signals comprises the physiological variability (PV), which reflect the variability of the cardiac frequency (CF) and of the mean arterial pressure (mAP) of the patient, the at least one index calculated from the signals representative of the respiratory activity comprises the autonomy index (AI), which reflect the percentage of total respiratory activity autonomously driven by the patient, the discomfort index (DI), which reflects the discomfort of the patient under respiratory assistance, and/or the respiratory variability (RV) which reflects the variability of the mean volume of air (MVA) and the mean respiratory frequency (MRF) signals, and the at least one index representative of the motor activity comprises the movement index (MI), reflecting the recovery of consciousness and/or agitation of the patient; —the global index is also determined from an additional index representative of the reactivity of the patient (RE), and reflecting the reactivity of the patient to the environment; —the signals representative of the cardio-circulatory state of the patient comprise heart rate, systolic blood pressure, diastolic blood pressure and/or mean arterial pressure; —the signals representative of the respiratory activity of the patient comprise signals representative of oxygen pulsed saturation, signals from a capnogram and/or signals from a respirator ensuring the artificial ventilation of the patient; —the signals from the respirator ensuring the artificial ventilation of the patient comprise signals relating to the airways pressure, the volume ventilated per minute and/or the respiratory rate; —the signals representative of the patient's motor activity are signals from an accelerometer; —the accelerometer is positioned on a distal segment of an upper limb and/or a lower limb of the patient and in that it records the acceleration of said limb continuously; —the signals representative of the cardio-circulatory state, the respiratory activity and the motor activity of the patient comprise artifacts, said artefacts being eliminated from said signals; —the signals representative of the cardio-circulatory state, the respiratory activity and the motor activity of the patient are filtered so as to obtain an averaged signal, in that the averaged signal is centered, and in that the variance of the centered average signal is calculated; —a time-frequency analysis of the signals representative of the cardio-circulatory state, the respiratory activity and the motor activity of the patient is carried out; —the frequencies of the signals representative of the cardio-circulatory state and/or the respiratory activity of the patient are sampled in frequency bands; —a database of sedated patients is provided, comprising continuous cardio-circulatory, respiratory and motor activity data; and—the calculated indexes and the global index are displayed on a device.

### BRIEF DESCRIPTION OF THE FIGURES

Other features and aspects of the present invention will be apparent from the following description and the accompanying drawings, in which:

FIG. 1 is a schematic view of the method according to the invention;

FIG. 2A shows an example of various elements that may be displayed on the device according to the invention, including a spider plot, a level indicator of the global sedation index, and an indicator of the trend of said index over the last hour, and FIG. 2B shows a curve that is illustrating the variation of the global sedation index over the last 12 hours, that may also be displayed on said device; and

FIGS. 3A and 3B compares the variation of the global sedation index with measurements on the RASS scale.

### DETAILED DESCRIPTION OF THE INVENTION

The invention relates to a method for automatic and continuous measurement of a state of sedation of a patient in an intensive care unit. Only these patients are specifically concerned with the subject of the invention. Patients who are not admitted to an intensive care unit and, in particular, patients under general anesthesia, are not concerned by the subject of the invention.

Sedation is a medical condition that focuses on the management of patients who are or may have one or more acute life-threatening disorders. It involves continuous monitoring of patients' vital functions and, when appropriate, the use of supplemental methods such as transfusion of blood derivatives, vascular filling, mechanical ventilation, catecholamines, hemodialysis and extracorporeal circulation. The ultimate goal of intensive care is the restoration of homeostasis. Sedation is a mean to facilitate the management of such ICU patients.

Upon arrival in an intensive care unit, the patient is connected to a multiparameter surveillance monitor. This monitor centralizes the measurements of various physiological variables of the patient. Among these signals extracted from the monitor, the invention thrives on:
electrocardiogram (ECG) measurements; and
arterial line measurements.

The ECG is, for example, a graphical representation of the electrical activity of the heart of a patient, over a period of time. The ECG may be obtained by using electrodes placed over the skin of the patient.

An arterial line is, for example, a thin catheter inserted into an artery, used to monitor various signals related to blood pressure, directly and in real-time (rather than by intermittent and indirect measurement), and to obtain blood samples for arterial blood gases analysis. The various signals related to blood pressure may be used for the implementation of the method of the invention.

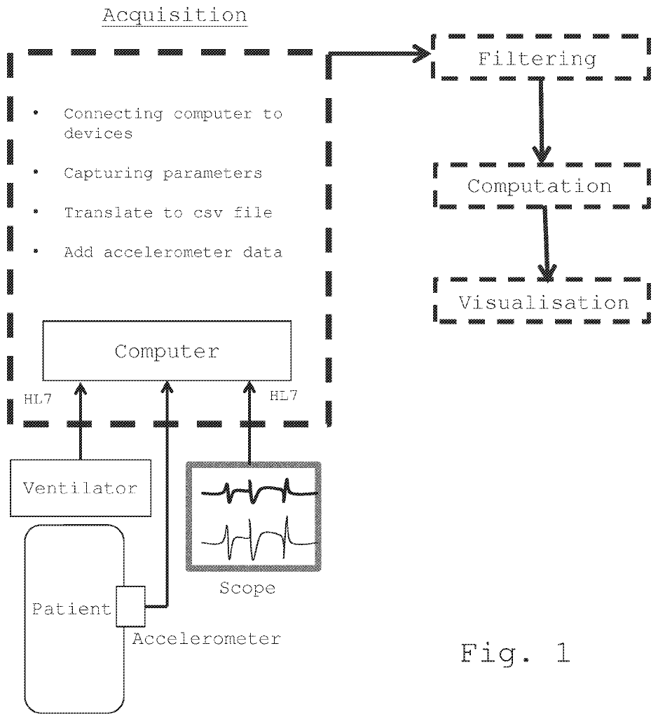

As shown in FIG. 1, besides the monitor measurements, respiratory parameters are extracted directly from the ventilator measurements.

The ventilator is, for example, a ventilator that comprises a tube that is placed in the mouth, the nose, or through a small cut in the throat of the patient, which ensures mechanical ventilation of said patient with a view to help this patient breathing. In addition, the ventilator provides various signals that are used for the implementation of the method of the invention.

The monitor centralizes the data contained in the various measurements and makes alleged real-time signals recordings, for example, every second. The monitor send an information every s and the signal is captured through the RS232 plug of the monitor. A computer is connected to both the monitor and the ventilator and capture the information sent by the two devices. These information are in generic format called HL7, which does not depend on the manufacturer of the monitor.

Additionally, means for measuring motor activity of the patient is used in order to detect a movement from the patient. The means for measuring motor activity is preferentially an accelerometer but can be chosen from any means for measuring motor activity such as an infrared sensor, a pressure sensor or the like. The means for measuring motor activity is positioned on the distal segment of an upper and/or a lower limb of the patient. This means for measuring motor activity is connected to the same computer (PC) where measurements are collected and stored in the same file than the monitor and ventilator parameters. In a preferred embodiment, the means for measuring motor activity is an accelerometer positioned on the distal segment of an upper and/or a lower limb of the patient. This accelerometer is connected to the same computer (PC) where measurements are collected and stored in the same file than the monitor and ventilator parameters.

According to a first step of the method of the invention, signals representing the state or condition of the patient are provided. These signals consist solely of cardio-circulatory signals, signals representative of respiratory activity and signals representative of the motor activity of the patient.

It will be noted that, advantageously, the signals other than those mentioned above are not taken into account in the method according to the invention. In particular, electroencephalographic signals are excluded from consideration. Indeed, taking into account the electroencephalographic signals may lead to errors in measuring the sedation state of the patients. As a matter of fact, patients are sedated patients, in an intensive care unit. They are not under general anesthesia. Finally, consideration of only the three aforementioned groups of signals, i.e. a minimum of groups of signals, leads, as will be explained hereinafter in the present description, to the construction of a particularly relevant index for measuring the sedation state of a patient.

The signals representative of the cardiovascular condition of the patient are issued from the ECG and the arterial line. From the ECG, the cardiac frequency (CF) is extracted. From the arterial line, the systolic (SBP), the diastolic (DBP) and the mean arterial pressure (mAP) are extracted. The signals representative of the respiratory activity of the patient are issued from the ventilator, and include 4 variables. The mean volume of air (MVA) is a variable indicating the total volume of air-oxygen mixture entering the respiratory system of patients. MVA reflects the volume of air furnished to patients by the ventilator device, added to the spontaneous volume of air (SpVA) autonomously consumed by patients. Similarly, the mean respiratory frequency (MRF) is a variable indicating the frequency at which air is entering the respiratory system of patients. MRF reflects the frequency of the mechanically ventilated breath, added to the spontaneous and autonomous frequency of breathing originating from patients (SpRF). We therefore get four variables from the ventilator device: MVA, SpVA, MRF, and SpRF. Signals representative of the patient's motor activity (ACC) are signals that are issued from a means for measuring motor activity. In a particular embodiment, the means for measuring motor activity is an accelerometer. It will be noted that such a means for measuring motor activity, more particularly such an accelerometer, is positioned on the distal segment of an upper limb and/or a lower limb of the patient, and records the movement, in particular the acceleration, of said limb continuously.

The data contained in the cardio-circulatory signals and, advantageously, those representative of the respiratory activity of the patient are generated by the multiparametric monitor. These information are recovered, for example, by means of an RS232 cable connected, on the one hand, to the monitor and, on the other hand, to a personal computer (PC) comprising software means for capturing such information. In their raw form, the HL7 files are not directly exploitable. They are therefore to be treated with a view to extracting the data necessary for their use according to the invention. The software means for processing the HL7 files include a routine, developed for example under the computer language Python™, making it possible to reformat the data contained in the HL7 files into a chronological text file under the CSV format (Comma-Separated Values), which allows analysis of the signals. It should be noted that the PC used to run this routine is also collecting ACC incoming from the means for measuring motor activity, preferentially incoming from the accelerometer. Therefore, ACC signal is concatenated to the CSV file together with the ventilator parameters.

Additionally, clinical data entered by the healthcare staff are synchronously collected with the acquisition of all the data described above, and added to the CSV file every 2 hours. These clinical data include age, sex, and data on the specialized care actually performed.

After data acquisition, the signals representative of the cardio-circulatory state, the respiratory activity and the motor activity of the patient are filtered so as to obtain an averaged signal, in that the averaged signal is centered, and in that the variance of the centered average signal is calculated. Data computation, including data processing and estimation of the sedative states of the patients then occurs in two steps. First, several single indices are calculated from variables collected by the ventilator, the physiological device and the means for measuring motor activity, in particular the accelerometer. These indices are subsequently integrated within GSI reflecting sedative states of patients. Single indices and GSI are normalized metrics varying between 0 and 1, with 0 associated to deep sedative states and 1 associated to recovery of consciousness.

Table 1 hereunder provides an overview of these indices, inputs they are derived from, and indicate if normalization is achieved solely through signal processing, or if an additional statistical step (i.e. logistic regression) is required.

TABLE 1

| | | Metric | |
|---|---|---|---|
| | | normalized | |
| | | after | Statistical |
| | | signal | normalization |
| | | processing | required |
| Index | Input devices | (YES/NO) | (YES/NO) |
| Autonomy | Ventilator | YES | NO |
| Index (AI) | | | |
| Discomfort | | YES | NO |
| Index (DI) | | | |
| Respiratory | | NO | YES |
| Variability | | | |
| (RV) | | | |
| Physiological | Physiological | NO | YES |
| Variability | device | | |
| (PV) | | | |
| Movement | Accelerometer | NO | YES |
| index (MI) | | | |

It is noted that the rationale of the Autonomy Index (AI) is straightforward: air consumption of the patients into deep sedative states solely relies on the ventilator device, while patients recovering consciousness tend to manifest signs of respiratory autonomy. Therefore, AI reflects the percentage of total respiratory activity (MVA and MRF) autonomously driven by patients (SpVA and SpRF). AI is computed through the formula:

$$AI = \frac{1}{2} * ( \frac{SpVA}{MVA} + \frac{SpRF}{MRF} )$$

AI is therefore normalized and varies between 0 and 1, with 0 associated to deep sedative state, and 1 associated to consciousness.

The Discomfort Index (DI) relies on the assumption that the respiratory assistance that is provided by the ventilator, is a source of discomfort when reaching consciousness. Clinical signs of discomfort manifest in excessive SpVA and SpRF. Therefore, DI is estimated using raw values of SpVA and SpRF, respectively associated to thresholds of discomfort derived from clinical expertise.

SpVA is estimated as follows:

$$f ( SpVA ) = \{ \begin{matrix} & 0 , SpVA < 15 \\ \frac{SpVA - 15}{5} , & ( SpVA \geq 15 ) and ( SpVA \leq 20 ) \\ & 1 , SpVA > 20 \end{matrix}$$

SpRF is estimated as follows:

$$f ( SpRF ) = \{ \begin{matrix} & 0 , SpRF < 20 \\ \frac{SpRF - 20}{20} , & ( SpVA \geq 20 ) and ( SpRF \leq 40 ) \\ & 1 , SpVA > 40 \end{matrix}$$

Then, DI is computed through the formula:

DI=½*(f(SpVA)+f(SpRF))

DI is therefore normalized and varies from 0 to 1, 0 being associated with deep sedative states, and 1 with signs of respiratory discomfort occurring when patients recover consciousness.

The Respiratory Variability (RV) is an index reflecting the variability of MVA and MRF. Patients under deep sedative state usually fully rely on the ventilator device for breathing, which is set up at a fixed volume and fixed frequency by the medical team. The rationale of estimating the variability of RV lies upon the assumption that variability of MVA and MRF is low when associated to deep sedative states, while this variability increase when reaching consciousness. In order to estimate the variability of MVA and MRF, we use four computational steps. Firstly, the log returns for every data point i of signals MVA and MRF are estimated through the formula:

$$logReturn ( MVA ( i ) ) = \{ \begin{matrix}0 , if MVA ( i ) = 0 \\ 0 , if MVA ( i - 1 ) = 0 \\ log ( \frac{MVA ( i )}{MVA ( i - 1 )} ) , if MVA ( i ) \neq 0 and MVA ( i - 1 ) \neq 0\end{matrix} \\ logReturn ( MRF ( i ) ) = \{ \begin{matrix}0 , if MRF ( i ) = 0 \\ 0 , if MRF ( i - 1 ) = 0 \\ log ( \frac{MRF ( i )}{MRF ( i - 1 )} ) , if MRF ( i ) \neq 0 and MRF ( i - 1 ) \neq 0\end{matrix}$$

Secondly, over moving sliding windows of 15 minutes (900 sampling points at with a frequency rate of 1 Hz), the arc length of signals log Return(MVA) and log Return(MRF) are linearly approximated through the formula:

$$Arclength ( MVA ( i ) ) = {\int }_{i - 900}^{i} \sqrt{1 + {( \frac{dlogReturn ( MVA )}{dt} )}^{2}} dt$$

$$Arclength ( MRF ( i ) ) = {\int }_{i - 900}^{i} \sqrt{1 + {( \frac{dlogReturn ( MRF )}{dt} )}^{2}} dt$$

Thirdly, the arc length of RV(i) is computed as follows:

Arclength(RV(i))=½(Arclength(MVA(i))+Arclength(MRF(i)))

Finally, the Arclength(RV) is normalized. Normalization is achieved through a simple logistic regression model, estimating the probability of RV being associated to recovery of consciousness (i.e. varying from 0 to 1). Form of the logistic regression model, estimating the probability of a patient reaching consciousness from Arclength(RV) at sampling point i is:

$$RV ( i ) = \frac{1}{1 + {e}^{- ( {β}_{0} + {β}_{1} Arclength ( RV ( i ) ) )}}$$

The intercept (β₀) and slope (β₁) of this model are estimated by using data collected in the intensive care unit of the Timone Hospital, Marseille, France.

Logistic regression therefore allows to estimate RV(i) as a metric varying from 0 to 1, with 0 associated to deep sedative states, and 1 with signs of recovery of consciousness.

The Physiological Variability (PV) is an index reflecting the variability of CF and mAP. Estimation of PV is similar to estimation of RV, relying on the assumption that CF and mAP increases when switching from deep sedative states to consciousness. Hence, computation of PV is similar to the computation of RV, with four computational steps. Firstly, it is estimated that the log returns for every data point i of signals CF and mAP through the formula:

$$logReturn ( CF ( i ) ) = \{ \begin{matrix}0 , if CF ( i ) = 0 \\ 0 , if CF ( i - 1 ) = 0 \\ log ( \frac{CF ( i )}{CF ( i - 1 )} ) , if CF ( i ) \neq 0 and CF ( i - 1 ) \neq 0\end{matrix} \\ logReturn ( mAP ( i ) ) = \{ \begin{matrix}0 , if mAP ( i ) = 0 \\ 0 , if mAP ( i - 1 ) = 0 \\ log ( \frac{mAP ( i )}{mAP ( i - 1 )} ) , if mAP ( i ) \neq 0 and mAP ( i - 1 ) \neq 0\end{matrix}$$

Secondly, over moving sliding windows of 15 minutes (900 sampling points at with a frequency rate of 1 Hz), the arc length of signals log Return(CF) and log Return(mAP) are linearly approximated through the formula:

$$Arclength ( CF ( i ) ) = {\int }_{i - 900}^{i} \sqrt{1 + {( \frac{dlogReturn ( CF )}{dt} )}^{2}} dt$$

$$Arclength ( mAP ( i ) ) = {\int }_{i - 900}^{i} \sqrt{1 + {( \frac{dlogReturn ( mAP )}{dt} )}^{2}} dt$$

Thirdly, the arc length of PV(i) is computed as follows:

Arclength(PV(i))=½*(Arclength(CF(i))+Arclength(mAP(i)))

Finally, Arclength(PV) is normalized. The normalization is achieved through a simple logistic regression model, estimating the probability of PV being associated to recovery of consciousness (i.e. varying from 0 to 1). Form of the logistic regression model, estimating the probability of a patient reaching consciousness from Arclength(PV) at sampling point i is:

$$PV ( i ) = \frac{1}{1 + {e}^{- ( {β}_{0} + {β}_{1} Arclength ( PV ( i ) ) )}}$$

Intercept (β₀) and slope (β₁) of this model are estimated by using data collected in the intensive care unit of the Timone Hospital, Marseille, France.

Logistic regression therefore allows to estimate PV(i) as a metric varying between 0 and 1, with 0 associated to deep sedative states, and 1 with signs of recovery of consciousness.

The Movement Index (MI) is estimated by using ACC values. Fluctuations of ACC indicate movements occurring on the hand where the accelerometer is located, therefore associated to recovery of consciousness and possibly agitation. Raw ACC values are directly used, but they are normalized through a logistic regression model such as:

$$MI ( i ) = \frac{1}{1 + {e}^{- ( {β}_{0} + {β}_{1} ( ACC ( i ) ) )}}$$

Intercept (β₀) and slope (β₁) of this model are estimated by using data collected in the intensive care unit of the Timone Hospital, Marseille, France.

Logistic regression therefore allows to estimate MI(i) as a metric varying from 0 to 1, with 0 associated to deep sedative states, and 1 with signs of recovery of consciousness.

Finally, an additional index RE may be added, that combines CF, MAP, the Respiratory rate and the Airway pressure. This index reflects the reactivity of these parameters: it computes the envelope of all the above signals and then compute the ratio between the lower envelope that is the resting state and the amplitude of the fluctuation that is the difference between the lower and the higher envelope. This ratio indicates how far these parameters are from the resting state. All the ratio are multiplied in order to enhance a coincidence between all the set of parameters. This index reflects therefore the reactivity of the patient to all external stimuli.

The GSI is derived from the single indices AI, DI, RV, PV, MI and RE, by estimating the probability of a patient reaching consciousness through a multivariate linear regression integrating these indices, such as, for every single sampling point:

$$GI ( i ) = \frac{1}{1 + {e}^{- ( {β}_{0} + {β}_{1} AI ( i ) + {β}_{2} DI ( i ) + {β}_{3} RV ( i ) + {β}_{4} PV ( i ) + {β}_{5} MI ( i ) ) + {β}_{6} RE ( i )}}$$

Coefficients of this model are estimated by using data collected in the intensive care unit of the Timone Hospital, Marseille, France.

Logistic regression therefore allows to estimate GI(i) as a global metric varying from 0 to 1, with 0 associated to deep sedative states, and 1 with signs of recovery of consciousness.

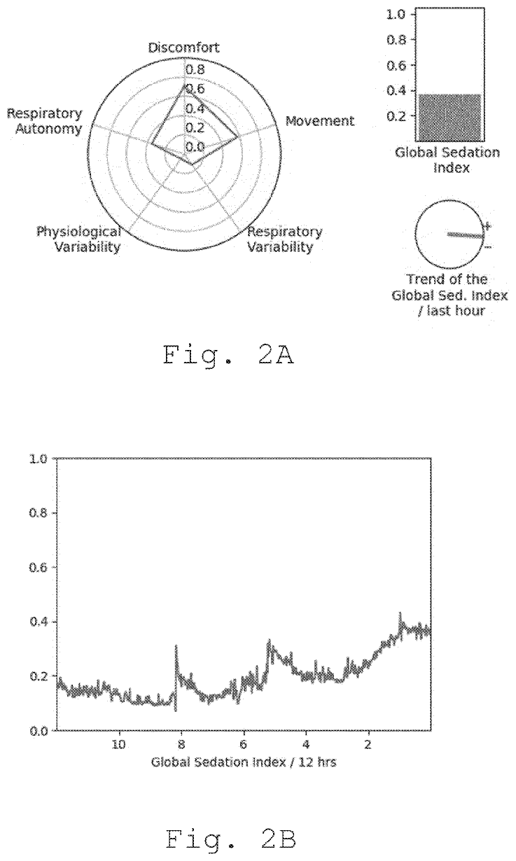

For data visualization, single indices and GSI described above are displayed on the same device used to gather data and compute these indices. A spider plot as shown in FIG. 2A is advantageously used to display values of the single indices. The GSI level is advantageously displayed next to the spider plot, as well the trend of said index over the last hour. Additionally, the variation of the GSI over the last 12 hours may be displayed, as sown in FIG. 2B.

Finally, the approach that is carried out according to the present invention is pragmatic. It uses: (1) adaptation to the respirator, (2) mastered motor skills (absence of agitation but persistence of motor reactivity), and (3) stabilization of cardio-circulatory parameters, that features that are provided in intensive care units. The index therefore makes it possible to quantify these three classes of parameters in order to make them measurable and to be able to disrupt them so as to make the patient more adapted to the environment of the intensive care unit, thus ensuring accurate medical management and comfort of the patient.

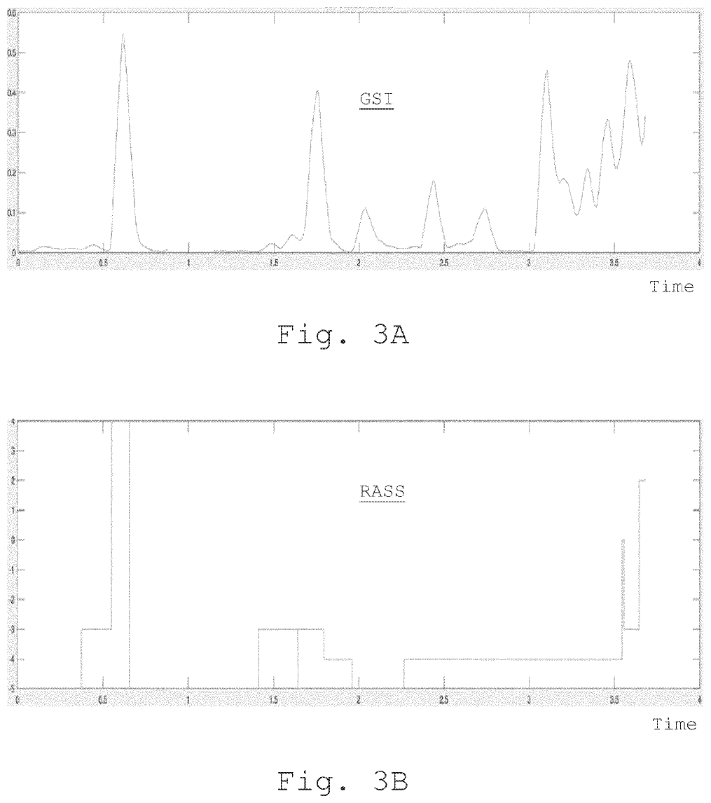

FIGS. 3A and 3B compare the variation of the global sedation index with measurements on the RASS scale for a particular patient. As shown in these figures, the variation of the GSI globally meets the variation of the RASS. Variations of the GSI as shown in FIG. 3A are however provided in real time, and are more precise.

1. A method for automatic and continuous measurement of a sedation state of a patient in an intensive care unit, the method comprising:
providing a multiparameter surveillance monitor centralizing measurements of various physiological variables of the patient including electrocardiogram measurements;
providing signals representative of a condition of the patient, the signals comprising cardio-circulatory signals, signals representative of a respiratory activity and signals representative of a motor activity of the patient; and
determining a global index representative of the sedation state of the patient,
wherein the cardio-circulatory signals are provided by a multiparameter surveillance monitor, the signals representative of the respiratory activity of the patient are provided by a ventilator, and the signals representative of the motor activity are provided by means for measuring motor activity of the patient.

2. The method according to claim 1, further comprising:
calculating, from the signals representative of the condition of the patient, at least one index calculated from the cardio-circulatory signals, at least one index calculated from the signals representative of the respiratory activity, and at least one index representative of the motor activity of the patient; and
determining, from these indexes, the global index representative of the sedation state of the patient.

3. The method according to claim 2, wherein
the at least one index calculated from the cardio-circulatory signals comprises a physiological variability (PV), which reflect a variability of the cardiac frequency (CF) and of a mean arterial pressure (mAP) of the patient,
the at least one index calculated from the signals representative of the respiratory activity comprises at least one selected from the group consisting of an autonomy index (AI), which reflects a percentage of total respiratory activity autonomously driven by the patient, a discomfort index (DI), which reflects a discomfort of the patient under respiratory assistance, and a respiratory variability (RV), which reflects a variability of a mean volume of air (MVA) signal and a mean respiratory frequency (MRF) signal, and
the at least one index representative of the motor activity comprises a movement index (MI), which reflects at least one selected from the group consisting of recovery of consciousness and agitation of the patient.

4. The method according to claim 2, wherein the global index is also determined from an additional index representative of a reactivity of the patient (RE), which reflects a reactivity of the patient to the environment.

5. The method according to claim 1, wherein the signals representative of the cardio-circulatory state of the patient comprise at least one selected from the group consisting of heart rate, systolic blood pressure, diastolic blood pressure, and mean arterial pressure.

6. The method according to claim 1, wherein the signals representative of the respiratory activity of the patient comprise at least one selected from the group consisting of signals representative of oxygen pulsed saturation, signals from a capnogram, and signals from a respirator ensuring an artificial ventilation of the patient.

7. The method according to claim 6, wherein the signals from the respirator ensuring the artificial ventilation of the patient comprise signals relating to at least one selected from the group consisting of airways pressure, a volume ventilated per minute, and a respiratory rate.

8. The method according to claim 1, wherein the signals representative of the patient's motor activity are signals from an accelerometer.

9. The method according to claim 8, wherein the accelerometer is positioned on a distal segment of an upper limb or a lower limb of the patient and records an acceleration of the limb continuously.

10. The method according to claim 1, comprising eliminating artifacts from the signals representative of the cardio-circulatory state, the respiratory activity and the motor activity of the patient.

11. The method according to claim 1, comprising filtering the signals representative of the cardio-circulatory state, the respiratory activity and the motor activity of the patient so as to obtain an averaged signal, centering the averaged signal so as to obtain a centered average signal, and calculating a variance of the centered average signal.

12. The method according to claim 1, comprising carrying out a time-frequency analysis of the signals representative of the cardio-circulatory state, the respiratory activity and the motor activity of the patient.

13. The method according to claim 1, comprising sampling frequencies of signals in frequency bands, wherein the signals are at least one selected from the group consisting of the signals representative of the cardio-circulatory state and the signals representative of the respiratory activity of the patient.

14. The method according to claim 1, comprising providing a database of sedated patients, the database comprising continuous cardio-circulatory, respiratory and motor activity data.

15. The method according to claim 1, comprising displaying the calculated indexes and the global index on a device.

16. The method according to claim 3, wherein the global index is also determined from an additional index representative of a reactivity of the patient (RE), which reflects a reactivity of the patient to the environment.

17. The method according to claim 2, wherein the signals representative of the cardio-circulatory state of the patient comprise at least one selected from the group consisting of heart rate, systolic blood pressure, diastolic blood pressure, and mean arterial pressure.

18. The method according to claim 3, wherein the signals representative of the cardio-circulatory state of the patient comprise at least one selected from the group consisting of heart rate, systolic blood pressure, diastolic blood pressure, and mean arterial pressure.

19. The method according to claim 4, wherein the signals representative of the cardio-circulatory state of the patient comprise at least one selected from the group consisting of heart rate, systolic blood pressure, diastolic blood pressure, and mean arterial pressure.

20. The method according to claim 2, wherein the signals representative of the respiratory activity of the patient comprise at least one selected from the group consisting of signals representative of oxygen pulsed saturation, signals from a capnogram, and signals from a respirator ensuring an artificial ventilation of the patient.
